(19) United States
(12) Patent Application Publication
(10) Pub. No.: US 2025/0229000 A1
(43) Pub. Date: Jul. 17, 2025

(54) ACELLULAR DERMAL TISSUE PREPARED USING SUPERCRITICAL FLUID EXTRACTION, AND USE THEREOF

(71) Applicant: DOF INC., Hwaseong-si (KR)

(72) Inventors: Yong Woo SHIN, Hwaseong-si (KR); Seong-Rae NOH, Hwaseong-si (KR)

(21) Appl. No.: 18/854,673

(22) PCT Filed: Feb. 23, 2023
(86) PCT No.: PCT/KR2023/002604

§ 371 Date: Oct. 7, 2024

(30) Foreign Application Priority Data
Apr. 8, 2022 (KR) 10-2022-0043858

(51) Int. Cl. A61L 27/36

(52) U.S. Cl. CPC A61L 27/3687; A61L 27/362

(57) ABSTRACT

The present invention relates to a decellularized acellular dermal tissue prepared using supercritical fluid extraction. More specifically, the present invention relates to an acellular dermal tissue having an improved level of extracellular matrix preservation and optimized mechanical properties such as critical load and elastic restoring force by using supercritical fluid extraction. The acellular dermal tissue according to the present invention is obtained through decellularization without a surfactant treatment so that mechanical properties are maintained, does not possess toxicity resulting from residual surfactants and minimizes transplant rejection, and thus can be effectively used for treatment of patients with damaged skin tissue.

### TECHNICAL FIELD

The present invention relates to an acellular dermal tissue prepared using supercritical fluid extraction. More specifically, the present invention relates to an acellular dermal tissue having an improved level of extracellular matrix preservation and optimized mechanical properties such as critical load and elastic restoring force by using supercritical fluid extraction.

### BACKGROUND ART

The skin tissue of the human body is composed of an epidermal layer, which is an outermost layer, a dermal layer below it, and a subcutaneous tissue. Among these, the epidermal layer is comprised of epithelial cells differentiated into several layers from the basement membrane that allows the epidermal layer and the dermal layer to be firmly bound, and melanocytes, and immune cells. The dermal layer below the epidermal layer is mainly comprised of fibroblasts and an extracellular matrix composed of collagen and elastin, etc.

Skin tissue or internal organ tissue may be partially damaged due to burns, trauma, ulcers, and the like. In this case, a method of transplanting one's own skin tissue or internal organ tissue is used for the purpose of healing the damaged tissue or for the purpose of reconstructive plastic surgery. In such cases, the recipient has to undergo the surgical burden of additionally extracting his or her own skin tissue or organ tissue, which can be dangerous if the recipient is not in good health. In addition, there are methods of transplanting using xenografts or synthetic biomaterials. However, these methods can cause immune rejection reactions, which can lead to long-term inflammatory reactions upon transplantation, resulting in the need for reoperation.

In order to solve the above problems, methods for preparing an acellular dermal layer for transplantation from skin tissue extracted from a donor are disclosed in Korean Patent Registration Nos. 10-0469661 and 10-0791502. However, these methods follow a surfactant treatment process. Surfactant treatment may cause problems such as denaturation of proteins such as collagen and destruction of growth factors. In addition, it is difficult to maintain the structural morphology and histological morphology, and mechanical properties such as critical load and elasticity may be weakened. Furthermore, it is not easy to remove the residual surfactant, and if it is not completely removed, it may be toxic.

In addition, in order to prepare an acellular dermal matrix, proteolytic enzymes such as trypsin or dispase have been used as immunogenic cell removal methods, or methods such as repeated freezing-thawing, NaCl and SDS treatment have been used. However, an acellular dermal matrix prepared by these processes has many antigen components, so it causes an immune rejection reaction when transplanted to the recipient site, resulting in a low engraftment rate (R. J. Walter et. al, Burns, 24: 104-113, 1998).

### DETAILED DESCRIPTION OF INVENTION

### Technical Problem

Accordingly, the present inventors conducted research to develop an acellular dermal graft material that improves the problems of transplant rejection and cost, while also ameliorating the problem of weakened mechanical properties caused by decellularization by chemical treatment. As a result, an optimized supercritical fluid treatment process was established without surfactant treatment, and an acellular dermal tissue having improved mechanical properties and improved levels of extracellular matrix preservation was obtained. Based on the above, the present inventors completed the present invention.

### Solution to Problem

In order to solve the above object, in one aspect of the present invention, there is provided an acellular dermal tissue graft, wherein a critical load of the acellular dermal tissue is 11 to 19 N, and a Young's modulus of the acellular dermal tissue is 0.5 to 1.2 MPa.

In another aspect of the present invention, there is provided a method for preparing an acellular dermal tissue graft, comprising the steps of a) separating skin tissue separated from a subject into an epidermal layer and a dermal layer; b) extracting the separated dermal layer with a supercritical fluid; and c) washing the dermal layer extracted with a supercritical fluid with a phosphate buffer.

### Effects of Invention

The acellular dermal tissue according to the present invention is obtained through decellularization without a surfactant treatment so that mechanical properties such as critical load and elasticity are maintained, and at the same time, does not possess toxicity resulting from residual surfactants, so it is suitable for transplantation to patients with damaged skin tissue such as burns and trauma. Therefore, the acellular dermal tissue of the present invention obtained through an optimized supercritical fluid treatment process does not possess toxicity and transplant rejection, and thus can be effectively used for treatment of patients with damaged skin tissue.

### BRIEF DESCRIPTION OF DRAWINGS

FIG. 1 is a photograph observing the appearance of the acellular dermal matrix sample according to one embodiment of the present invention. Here, the RTU (Ready-To-Use) type is prepared by double-packaging and sterilizing a dermal sample including normal saline. The FD (Feeze-Drying) type is prepared by freeze-drying a dermal sample, and then double-packaging and sterilizing it. In addition, Native is an unprocessed natural skin tissue sample without removing the epidermis, and SCR, SCF, A, B, C, D, E, and F are as shown in Table 1 below. The size of the dermal sample is 1×1 cm².

FIG. 2 is a microscope photograph (100× magnification) showing the results of H&E (Hematoxyline & Eosin) staining an acellular dermal matrix sample obtained after the supercritical fluid extraction process according to one embodiment of the present invention.

FIG. 3 is a 1% agarose gel electrophoresis image confirming an amount of residual DNA in a dermis tissue (Native) before the supercritical fluid extraction process according to one embodiment of the present invention, a decellularized dermal tissue (SCR and SCF) after the supercritical fluid extraction process, and an acellular dermal tissue (A, B, C, D, E, and F) prepared according to a conventionally known method according to the detailed specifications of Table 1. Here, SCR, A, B, and C are RTU (Ready-To-Use) type samples, and SCF, D, E, and F are FD (Feeze-Drying) type samples.

FIG. 4a is a drawing showing the results of quantitative analysis of residual DNA of an acellular dermal matrix sample obtained after the supercritical fluid extraction process according to one embodiment of the present invention. In this case, the results of quantitative analysis of residual DNA are shown based on the dry weight of the acellular dermal tissue. Here, Native indicates the original tissue as an untreated group, SCR and SCF are test groups of dermal tissue decellularized by treatment with a supercritical fluid, and A, B, C, D, E, and F are positive control groups of decellularized dermal tissue according to the detailed specifications of Table 1.

FIG. 4b is a drawing showing the results of quantitative analysis of residual DNA of an acellular dermal matrix sample obtained after the supercritical fluid extraction process according to one embodiment of the present invention. In this case, the results of quantitative analysis of residual DNA are shown based on the original tissue without removing an epidermal layer.

FIG. 5 is a drawing showing the results of Western blot analysis confirming the expression level of the immunogenic protein MHC1 in the acellular dermal matrix samples (SCR and SCF) obtained after the supercritical fluid extraction process according to one embodiment of the present invention.

FIG. 6a is a drawing showing the results of collagen content analysis of the acellular dermal matrix samples (SCR and SCF) obtained after the supercritical fluid extraction process according to one embodiment of the present invention. In this case, the results of collagen content analysis are shown based on the dry weight of the acellular dermal tissue.

FIG. 6b is a graph showing the results of quantitative analysis of the collagen content in the acellular dermal matrix samples (SCR and SCF) obtained after the supercritical fluid extraction process according to one embodiment of the present invention based on the original tissue.

FIG. 7a is a drawing showing the results of elastin content analysis of the acellular dermal matrix samples (SCR and SCF) obtained after the supercritical fluid extraction process according to one embodiment of the present invention. In this case, the results of elastin content analysis are shown based on the dry weight of the acellular dermal tissue.

FIG. 7b is a graph showing the results of quantitative analysis of the elastin content in the acellular dermal matrix samples (SCR and SCF) obtained after the supercritical fluid extraction process according to one embodiment of the present invention based on the original tissue.

FIG. 8a is a drawing showing the results of a cytokine array that confirms the level of growth factor preservation in an acellular dermal matrix sample obtained after the supercritical fluid extraction process according to one embodiment of the present invention.

FIG. 8b is a drawing showing the spot coordinates of a cytokine array that confirms the level of growth factor preservation in an acellular dermal matrix sample obtained after the supercritical fluid extraction process according to one embodiment of the present invention.

FIGS. 8c to 8f are graphs showing the results obtained by quantifying the cytokine array results of confirming the level of growth factor preservation in an acellular dermal matrix sample obtained after the supercritical fluid extraction process according to one embodiment of the present invention by mean spot pixel intensity.

FIG. 9 is a drawing showing the results of evaluation of the cytotoxicity of an acellular dermal matrix sample obtained after the supercritical fluid extraction process according to one embodiment of the present invention.

FIG. 10a is a graph showing the results obtained by measuring the critical load of an acellular dermal matrix sample obtained after the supercritical fluid extraction process according to one embodiment of the present invention using a universal tensile machine (UTM).

FIG. 10b is a graph showing the results of quantitative analysis of the critical load of an acellular dermal matrix sample obtained after the supercritical fluid extraction process according to one embodiment of the present invention based on the original tissue.

FIG. 11a is a graph showing the results of the Young's modulus measurement of an acellular dermal matrix sample obtained after the supercritical fluid extraction process according to one embodiment of the present invention.

FIG. 11b is a graph showing the results of quantitative analysis of the Young's modulus of an acellular dermal matrix sample obtained after the supercritical fluid extraction process according to one embodiment of the present invention based on the original tissue.

### BEST MODE FOR CARRYING OUT THE INVENTION

### Acellular Dermal Tissue Graft

In one aspect of the present invention, there is provided an acellular dermal tissue graft having the following characteristics:
a critical load of the acellular dermal tissue is 11 to 19 N, and a Young's modulus of the acellular dermal tissue is 0.5 to 1.2 MPa.

As used herein, the term “dermis” refers to a structure in which collagenous fibers made of collagen protein are the main component, and elastic fibers made of elastin protein are woven in a mesh shape between them. The dermis covers most of the skin and provides nutrients to the epidermis, supports the epidermis, and protects the body from external damage. In addition, it has the ability to store water, has the function of regulating body temperature, acts as a receptor for sensation, and has the function of regenerating the skin by interacting with the epidermis.

As used herein, the term “acellular dermal matrix (ADM)” refers to a dermal layer matrix obtained from human or animal skin through acellularization technology, and means a bio-derived skin substitute in the form of an extracellular matrix (ECM) composed of collagen and elastin, etc.

The acellular dermal matrix is a biomaterial obtained by removing cells that can cause an immune response from skin separated from a subject, and can be used to restore skin by transplanting it to patients with skin defects caused by burns, traffic accidents, ulcers, and the like. In addition, an acellular dermal matrix, which is a skin tissue for human transplantation, may find its extended use in reconstruction of full-thickness skin, septal defect, and dural defect of the brain and spinal cord, as well as to reconstructive and cosmetic surgery including reconstruction of sunken scars, reconstruction of hemifacial atrophy, nipple reconstruction, and lip enlargement.

The acellular dermal matrix should be selectively free of only cellular antigens that are the objects of immune response while maintaining various structural proteins and components without damaging the three-dimensional structure of the dermal layer in the skin tissue.

In addition, the acellular dermal matrix should have appropriate elasticity and critical load so that it can be stably maintained even when the applied treatment area moves while being applied to damaged tissue or damaged skin to restore the damaged area. The acellular dermal matrix should be a material that does not damage normal tissues neighboring the treatment area and should be easy to treat.

In the present specification, the acellular dermal matrix may be used interchangeably with an acellular dermal tissue graft, an acellular dermal tissue, a decellularized dermis, a decellularized dermal tissue, or an acellular dermal tissue.

As used herein, the term “decellularization” refers to a new method of producing an artificial support by removing cells from an entire organ while maintaining the original structure of the intended transplant tissue or organ. In the decellularization process, cellular components are removed from the tissue, but the extracellular matrix and some growth factor proteins are preserved. Therefore, various extracellular matrix components including collagen, fibronectin, and elastin preserved in the decellularized tissue can enhance the survival, proliferation, and differentiation of cultured cells by providing a three-dimensional microenvironment similar to that in the intact tissue.

In the present invention, decellularization may be performed by supercritical fluid extraction without surfactant treatment, but is not limited thereto.

As used herein, the term “supercritical fluid extraction” or “supercritical extraction” refers to a method of separating materials using a supercritical fluid having intermediate nature between gas and liquid that exists above the critical point, i.e., the critical temperature and critical pressure. The above supercritical fluid extraction employs in a combined manner, the solvent extraction principle in which soluble components contained in the raw material are dissolved into the supercritical fluid due to the difference in solubility between the raw material for extraction and the supercritical fluid, and the distillation principle in which solute molecules contained in the raw material are transferred from the high-density condensed phase to the supercritical fluid in the low-density expanded phase, as an evaporation phenomenon.

As used herein, the term “supercritical fluid” refers to a substance that is a gas under normal conditions but is a fluid above its critical temperature and critical pressure. Suitable supercritical fluids for use in the present invention are not particularly limited, but include, for example, carbon dioxide, nitrogen, nitrous oxide, methane, ethylene, propane, and propylene. Preferably, carbon dioxide having the critical temperature of 31° C. and the critical pressure of 72.8 atm may be used.

In the present invention, decellularization may be performed by adding a ‘co-solvent’ in addition to the supercritical fluid during supercritical fluid extraction. The above co-solvent may be added for the purpose of increasing the extractability of the supercritical fluid, and improving the solubility therein, etc. Ethanol, methanol, petroleum ether, acetonitrile, hexane, and the like may be used as the co-solvent, which are not limited thereto. In this case, the co-solvent may preferably be ethanol.

The decellularized dermal tissue of the present invention may be derived from skin tissue separated from a subject. The subject may be a subject of the same species or a different species as the subject to which the dermal tissue is to be transplanted, and specifically may be a mammal including a human, a mouse, a rat, a murine, a monkey, a chimpanzee, an orangutan, a horse, a cow, a pig, a cat, a dog, and a rabbit, but is not limited thereto.

The acellular dermal tissue of the present invention may be one in which cells are removed by 90% or more, 91% or more, 92% or more, 93% or more, 94% or more, 95% or more, 96% or more, 97% or more, 98% or more, 99% or more, or 100%, compared to the original tissue separated from the subject, that is, compared to the tissue that was not subjected to decellularization.

As used herein, the term “critical load” refers to the limit load at which a structure or member becomes in a state such as destruction or excessive deformation. That is, it refers to the load at the starting point where deformation or cracking of the sample piece occurs during the indentation evaluation performed by applying a load through an indenter. It refers to the maximum stress until deformation or cracking of the sample piece occurs due to the critical load, and means the value obtained by dividing the maximum load until rupture by the original cross-sectional area of the test piece. The greater the critical load, the greater the force required to break the support, which may also mean that the dermal tissue can maintain its shape well when applied to damaged skin or damaged tissue, as well as during distribution and storage.

The critical load measured in the present invention can be measured using a universal tensile machine. In addition, in the present invention, the critical load of the acellular dermal tissue may be a wet critical load measured under conditions of being wetted with sterile distilled water, i.e., a wet state, but is not limited thereto.

In the present invention, the decellularized dermal tissue may have a critical load of 11 to 19 N based on the size of 1×1 cm² of the dermal tissue sample under wet conditions.

In one embodiment, an RTU (Ready-To-Use) type dermal tissue sample that was not subjected to freeze-drying may have a critical load of 11.0 N, 11.1 N, 11.2 N, 11.3 N, 11.4 N, 11.5 N, 11.6 N, 11.7 N, 11.8 N, 11.9 N, or 12.0 N, but is not limited thereto. In this case, the critical load is measured based on the size of the dermal tissue sample of 1×1 cm².

In addition, in one embodiment, an FD (Freeze-Drying) type dermal tissue sample that was subjected to freeze-drying may have a critical load of 16.0 to 19.0 N, 16.5 to 19.0 N, 17.0 to 19.0 N, 17.5 to 19.0 N, 18.0 to 19.0 N, 18.1 N, 18.2 N, 18.3 N, 18.4 N, 18.5 N, 18.6 N, 18.7 N, or 18.8 N, but is not limited thereto. In this case, the critical load is measured based on the size of the dermal tissue sample of 1×1 cm².

As used herein, the term “Young's modulus” is also referred to as ‘elastic modulus’ or ‘elastic coefficient’, and refers to the ratio of the pressure (stress) of an object to the deformation of the object. The Young's modulus is an elastic modulus that indicates the degree to which an object is deformed when pressure is applied, and the basic principle of measuring a Young's modulus is to utilize the property that an object undergoes elastic deformation when compressed or expanded and returns to its original shape when the load is removed. For example, more deformation occurs in a flexible object compared to a stiff object, and a high Young's modulus value means inelasticity or stiffness.

The Young's modulus measured in the present invention may be measured using a universal tensile machine. In addition, the Young's modulus of the dermal tissue in the present invention may be measured under wet conditions, but is not limited thereto.

In the present invention, the decellularized dermal tissue may have a Young's modulus of 0.5 to 1.2 MPa based on the size of the dermal tissue sample of 1×1 cm² under wet conditions.

In one embodiment, an RTU (Ready-To-Use) type dermal tissue sample that was not subjected to freeze-drying may have a Young's modulus of 0.50 MPa, 0.51 MPa, 0.52 MPa, 0.53 MPa, 0.54 MPa, 0.55 MPa, 0.56 MPa, 0.57 MPa, 0.58 MPa, 0.59 MPa, or 0.60 MPa, but is not limited thereto. In this case, the Young's modulus is measured based on the size of the dermal tissue sample of 1×1 cm².

In addition, in one embodiment, an FD (Freeze-Drying) type dermal tissue sample that was subjected to freeze-drying may have a Young's modulus of 1.00 MPa, 1.01 MPa, 1.02 MPa, 1.03 MPa, 1.04 MPa, 1.05 MPa, 1.06 MPa, 1.07 MPa, 1.08 MPa, 1.09 MPa, or 1.10 MPa, but is not limited thereto. In this case, the Young's modulus is measured based on the size of the dermal tissue sample of 1×1 cm².

The acellular dermal tissue according to the present invention may have any one characteristic selected from the following characteristics based on dry weight, but is not limited thereto:
an amount of residual DNA of 80 to 110 ng/mg;
a collagen content of 400 to 700 μg/mg; and
an elastin content of 13 to 19 μg/mg.

In the present invention, the dermal tissue may comprise an amount of residual DNA of 110 ng/mg or less based on dry weight. Preferably, it may comprise 80 to 110 ng/mg, 81 to 109 ng/mg, 82 to 108 ng/mg, 83 to 107 ng/mg, 84 to 106 ng/mg, 85 to 105 ng/mg, 86 to 104 ng/mg, or 87 to 103 ng/mg, but is not limited thereto.

In the present invention, the dermal tissue may comprise a collagen content of 400 g/mg or more based on dry weight. Preferably, it may comprise 400 to 700 μg/mg, 402 to 698 g/mg, 404 to 696 μg/mg, 406 to 694 μg/mg, 408 to 692 μg/mg, 410 to 690 μg/mg, 411 to 688 g/mg, 412 to 686 μg/mg, 413 to 684 μg/mg, 414 to 682 μg/mg, 415 to 680 μg/mg, 416 to 679 g/mg, 417 to 678 μg/mg, or 418 to 677 μg/mg, but is not limited thereto.

In the present invention, the dermal tissue may comprise an elastin content of 13 μg/mg or more based on dry weight. Preferably, it may comprise 13.0 to 19.0 μg/mg, 13.1 to 18.9 g/mg, 13.2 to 18.8 μg/mg, 13.3 to 18.7 μg/mg, 13.4 to 18.6 μg/mg, 13.5 to 18.5 μg/mg, 13.6 to 18.4 μg/mg, 13.7 to 18.3 μg/mg, 13.8 to 18.2 μg/mg, or 13.9 to 18.1 μg/mg, but is not limited thereto.

The acellular dermal tissue according to the present invention may have any one characteristic selected from the following characteristics compared to the original tissue, but is not limited thereto:
an amount of residual DNA of 5 to 8%;
a collagen content of 55 to 99%;
an elastin content of 65 to 95%;
a critical load of 90 to 165%; and
a Young's modulus of 80 to 200%.

In the present invention, the dermal tissue may comprise an amount of residual DNA of 8% or less compared to the original tissue, that is, compared to a dermal tissue before decellularization or a dermal tissue that was not subjected to decellularization. Preferably, it may comprise 5 to 8%, 5.1 to 7.8%, 5.2 to 7.6%, 5.3 to 7.6%, 5.4 to 7.4%, 5.5 to 7.4%, 5.6 to 7.2%, 5.7 to 7.0%, or 5.8 to 6.8%, but is not limited thereto.

In other words, the dermal tissue may be one in which 93% or more of the cells have been removed compared to the original tissue. Preferably, it may be one in which 93 to 98%, 94 to 98%, 95 to 98%, 96 to 98%, or 97 to 98% of the cells have been removed, but is not limited thereto.

The dermal tissue according to the present invention may be one in which extracellular matrix components, such as collagen and elastin, are maintained at a predetermined content compared to the original tissue.

Specifically, in the present invention, the dermal tissue may comprise a collagen content of 55% or more compared to the original tissue. Preferably, it may comprise a collagen content of 55 to 99%, 56 to 98%, 57 to 97%, or 58 to 96%, but is not limited thereto.

In the present invention, the dermal tissue may comprise an elastin content of 65% or more compared to the original tissue. Preferably, it may comprise an elastin content of 65 to 95%, 66 to 94%, 67 to 93%, 68 to 92%, 69 to 91%, or 70 to 90%, but is not limited thereto.

In the present invention, the dermal tissue may exhibit a critical load of 90 to 165% compared to the original tissue, but is not limited thereto.

In the present invention, the dermal tissue may exhibit a Young's modulus of 80 to 200% compared to the original tissue, but is not limited thereto.

The acellular dermal tissue according to the present invention may have any one characteristic selected from the following characteristics compared to the original tissue, but is not limited thereto:
an adiponectin content of 22 to 44%;
an apolipoprotein A1 content of 84 to 94%;
an angiogenin content of 19 to 38%;
an angiopoietin-2 content of 90 to 99%;
a brain-derived neurotrophic factor (BDNF) content of 73 to 99%;
a complement component C5/C5a content of 9 to 19%;
a CD30 content of 59 to 99%;
a CD40 ligand content of 6 to 28%;
a CD26 content of 2 to 5%;
an Emmprin content of 36 to 44%;
a CD105 content of 41 to 99%;
a Fas ligand content of 2 to 45%;
a FGF-2 content of 15 to 64%;
a FGF19 content of 56 to 98%;
an IFN-γ content of 63 to 99%;
an IL-1A (alpha) content of 65 to 99%;
an IL-1RA (alpha) content of 12 to 43%;
an IL-17 content of 82 to 99%;
a kallikrein (KLK) 3 content of 92 to 99%;
a MIF (Macrophage Migration Inhibitory Factor) content of 2 to 9%;
a MMP (Matrix Metalloproteinase Protein) 9 content of 83 to 99%;
a pentraxin (PTX) 3 content of 44 to 91%;
a retinol binding protein (RBP) 4 content of 8 to 36%;
a vitamin D binding protein (VDBP) content of 6 to 16%; and
a CD31 content of 67 to 99%.

In exemplary embodiments of the present invention, the dermal tissue does not comprise a surfactant. In the case of dermal tissue decellularized by conventional surfactant treatment, there were problems such as denaturation of proteins such as collagen and destruction of growth factors due to surfactant treatment. In addition, it was not easy to remove the residual surfactant, and there was a problem that toxicity may occur if it was not completely removed.

In addition, there was a difficulty in that the mechanical properties of the dermal tissue, such as the critical load and elasticity, were weakened due to the surfactant, and when the surfactant remained in the dermal tissue, it caused transplant rejection due to the toxic substance.

However, a decellularized dermal tissue obtained by the supercritical fluid process according to the present invention has the characteristics of decellularization at the same level as the dermal tissue decellularized by conventional surfactant treatment, and of maintaining the histological morphology and mechanical properties. In particular, the acellular dermal tissue obtained by the supercritical fluid process has the advantage of significantly excellent preservation of extracellular matrix (ECM) substances such as collagen and elastin within the dermal tissue compared to the dermal tissue treated with the surfactant.

Therefore, the acellular dermal tissue according to the present invention can be utilized as a graft material by decellularization without loss of the extracellular matrix while maintaining the histological morphology and mechanical properties.

### Preparation of Acellular Dermal Tissue Graft

The acellular dermal tissue graft according to the present invention may be prepared by a preparation method comprising the step of extracting dermal tissue separated from a subject with a supercritical fluid.

The above “decellularization”, “subject”, “dermal tissue”, and “supercritical fluid” are the same as described above.

In the present invention, the supercritical fluid can extract lipid components, specifically, phospholipid components, which are main components of cell membranes, in dermal tissue separated from a subject based on solubility to afford decellularization to prepare a decellularized dermal tissue.

The supercritical fluid may be selected from the group consisting of carbon dioxide gas, ammonia gas, nitrogen gas, nitrogen monoxide (NO) gas, nitrogen dioxide (NO₂) gas, nitrous oxide (N₂O) gas, sulfur dioxide gas, hydrogen gas, water vapor, saturated hydrocarbons, unsaturated hydrocarbons, aromatic compounds, and mixed gases thereof. Specifically, it may be carbon dioxide gas, but the type is not limited thereto as long as it is a supercritical fluid that can efficiently prepare dermal tissue by removing most of the cells of the dermal tissue while maintaining the mechanical properties as well as the structural form of the dermal tissue. When carbon dioxide gas is used as the supercritical fluid, carbon dioxide has a low critical temperature (31° C.) and critical pressure (73 bar), so it can be easily adjusted to supercritical conditions, and has the advantages of being widely present in nature, colorless, odorless, harmless to the human body, and chemically stable.

In the present invention, the supercritical extraction step may be performed under a pressure condition of 0 to 1000 bar, 30 to 900 bar, 60 to 800 bar, 90 to 700 bar, 120 to 600 bar, 150 to 500 bar, or 200 to 400 bar.

Specifically, the pressure of the supercritical extraction step may be 0 bar or more, 50 bar or more, 100 bar or more, 150 bar or more, 200 bar or more, 250 bar or more, 300 bar or more, 350 bar or more, 400 bar or more, 450 bar or more, 500 bar or more, 550 bar or more, 600 bar or more, 650 bar or more, 700 bar or more, 750 bar or more, 800 bar or more, 850 bar or more, 900 bar or more, or 950 bar or more, but is not limited thereto.

In addition, the pressure of the supercritical extraction step may be 1000 bar or less, 950 bar or less, 900 bar or less, 850 bar or less, 800 bar or less, 750 bar or less, 700 bar or less, 650 bar or less, 600 bar or less, 550 bar or less, 500 bar or less, 450 bar or less, 400 bar or less, 350 bar or less, 300 bar or less, 250 bar or less, 200 bar or less, 150 bar or less, 100 bar or less, or 50 bar or less, but is not limited thereto.

The pressure condition of the supercritical extraction step is not limited thereto as long as it is a condition that can efficiently prepare dermal tissue by removing most of the cells of the dermal tissue while preserving the mechanical properties of the dermal tissue and the structural form of the tissue.

In the above supercritical extraction step, in addition to the supercritical fluid, a co-solvent may be further included. The co-solvent may be at least one solvent selected from the group consisting of ethanol, water, methanol, hexane, petroleum ether, acetonitrile, acetone, ethyl acetate, and methylene chloride. Preferably, ethanol may be further included as a co-solvent.

The co-solvent is added for the purpose of increasing the extractability of the supercritical fluid and improving the solubility therein, etc., and extracts lipids in the separated dermal tissue, specifically, phospholipids of the cell membrane, thereby removing most of the cells of the dermal tissue. However, the type thereof is not particularly limited as long as the co-solvent preserves the mechanical properties of the dermal tissue and the structural form of the tissue.

In the present invention, the supercritical extraction step may be performed under a temperature condition of 31° C. to 40° C., 31° C. to 39° C., 32° C. to 38° C., 33° C. to 37° C., 34° C. to 36° C., or 35° C., but is not limited thereto.

In the present invention, the supercritical extraction step may be performed for less than 3 hours, but is not limited thereto. Preferably, it may be performed for 60 minutes to 180 minutes, 70 minutes to 170 minutes, 80 minutes to 160 minutes, 90 minutes to 150 minutes, 100 minutes to 140 minutes, 110 minutes to 130 minutes, or 120 minutes, but but is not limited thereto.

The acellular dermal tissue according to the present invention may be prepared by including, but not limited to, the step of separating an epidermal layer and a dermal layer before the step of extracting with a supercritical fluid.

The separation of the epidermal layer and the dermal layer may be performed by a method known in the art. In general, the separation of the epidermal layer and the dermal layer may be performed using various proteolytic enzymes, such as dispase, thermolysin, trypsin, and the like.

In addition, the epidermal layer and the dermal layer may be separated by changing the ionic strength of the solution. Specifically, the epidermal layer and the dermal layer may be separated by treating with a 1 M or higher sodium chloride (NaCl) solution or a 20 mM EDTA solution at 37° C. for 14 to 32 hours.

In one embodiment, the separation of the epidermal layer and the dermal layer may be performed by treating with 1 M NaCl for 24 hours under a temperature condition of 37° C., but is not limited thereto.

The acellular dermal tissue according to the present invention may be prepared by further comprising, but not limited to, any one of the following steps after the step of extracting with a supercritical fluid:
washing a dermal tissue with a phosphate buffer;
freeze-drying the dermal tissue; and
sterilizing the dermal tissue.

The residual solution and impurities present in the dermal tissue after the supercritical fluid extraction can be washed through washing with a phosphate buffer.

In one embodiment of the present invention, after supercritical fluid extraction, the decellularized dermal tissue was washed with PBS (phosphate buffered saline) at room temperature for 16 hours to remove residual solution and impurities.

The dermal tissue obtained after the supercritical fluid extraction can be preserved through freeze-drying until use.

During the freeze-drying, a cryoprotectant may be additionally included, but is not limited thereto. The cryoprotectant can prevent structural changes in the dermal layer tissue, as well as physical and chemical damage to the dermal layer tissue.

As the cryoprotectant, a cryoprotectant known in the art may be adopted. Exemplary cryoprotectants may be sugars and corresponding sugar alcohols. Specific examples of sugars and corresponding sugar alcohols include sucrose, maltitol, glucitol, lactitol, and isomaltulose. In addition, currently widely used cryoprotectants include DMSO, dextran, propylene glycol, glycerol, trehalose, polyethylene glycol, serum albumin, and the like.

The dermal tissue may be treated with the cryoprotectant so that the cryoprotectant can be sufficiently penetrated into the dermal tissue before freeze-drying, but is not limited thereto. Specifically, the tissue treated with the cryoprotectant may be stored under ultra-low temperature freezing conditions of about −70° C. or lower, preferably −40° C. to −70° C. In addition, the tissue treated with the cryoprotectant may be stored for 4 hours or more, preferably 4 to 48 hours.

The freeze-drying may be performed using a freeze-dryer for 24 to 48 hours, but is not limited thereto.

In one embodiment of the present invention, the dermal tissue obtained after the supercritical fluid extraction was treated and penetrated with maltitol for freeze-drying, and then frozen and stored at a temperature of about −70° C. or lower for at least 4 hours. After freezing, it was freeze-dried for about 24 to 48 hours using a freeze-dryer and preserved until use.

The dermal tissue obtained after the supercritical fluid extraction may be sterilized by irradiation. The irradiation range may be 10 to 30 kGy, but is not limited thereto.

In one embodiment of the present invention, the dermal tissue obtained after the supercritical fluid extraction was sterilized by irradiating it with 10 to 30 kGy of gamma rays after packaging was completed.

### Use of Acellular Dermal Tissue Graft

The acellular dermal tissue according to the present invention is obtained through decellularization without a surfactant treatment so that mechanical properties such as critical load and elasticity are maintained, and does not possess toxicity resulting from residual surfactants. Therefore, the dermal tissue of the present invention obtained through an optimized supercritical fluid treatment process does not possess toxicity and transplant rejection, and thus can be effectively used for reconstructive plastic surgery and cosmetic plastic surgery, including serious burns, trauma, correction of sunken scars, correction of hemifacial atrophy, nipple reconstruction, and lip enlargement, etc.

### Method for Preparing Acellular Dermal Tissue Graft

In another aspect of the present invention, there is provided a method for preparing an acellular dermal tissue graft, comprising the steps of a) separating skin tissue separated from a subject into an epidermal layer and a dermal layer; b) extracting the separated dermal layer with a supercritical fluid; and c) washing the dermal layer extracted with a supercritical fluid with a phosphate buffer.

The above “subject” and “supercritical fluid” are the same as described above.

The above step a) is a step of separating skin tissue separated from a subject into an epidermal layer and a dermal layer. Specifically, the separation of the epidermal layer and the dermal layer may be performed by a method known in the art. In general, the separation of the epidermal layer and the dermal layer may be performed using various proteolytic enzymes, such as dispase, thermolysin, trypsin, and the like. In addition, the epidermal layer and the dermal layer may be separated by changing the ionic strength of the solution. Specifically, the epidermal layer and the dermal layer may be separated by treating with a 1 M or higher sodium chloride (NaCl) solution or a 20 mM EDTA solution at 37° C. for 14 to 32 hours.

The above step b) is a step of extracting the dermal layer from which the epidermis layer has been separated using a supercritical fluid. Specifically, cellular components other than the extracellular matrix from the dermal layer, such as phospholipid components, which are the main components of cell membranes that induce immune responses, are extracted using a supercritical fluid to afford decellularization.

The type of the supercritical fluid, the pressure conditions of supercritical fluid extraction, the temperature conditions, and the execution time are the same as described above in ‘Preparation of acellular dermal tissue graft’.

In addition, the supercritical fluid in step b) may further comprise a co-solvent, but is not limited thereto. The co-solvent is added for the purpose of increasing the extractability of the supercritical fluid and improving the solubility therein, etc., and may extract cellular components other than the extracellular matrix from the separated dermal layer, thereby removing most of the cells of the dermal tissue.

The type of the co-solvent is not particularly limited as long as the co-solvent preserves the mechanical properties of the dermal tissue and the structural form of the tissue, and the co-solvent may be at least one solvent selected from the group consisting of, for example, ethanol, water, methanol, hexane, petroleum ether, acetonitrile, acetone, ethyl acetate, and methylene chloride. Preferably, ethanol may be further included as a co-solvent.

The above step c) is a step of washing the dermal layer extracted with a supercritical fluid with a phosphate buffer. The residual solution and impurities present in the dermal tissue after the supercritical fluid extraction can be washed through washing with a phosphate buffer.

In addition, the dermal tissue after the washing may be additionally subjected to any one of freeze-drying, sterilization, and sealing packaging, but is not limited thereto.

### MODE FOR CARRYING OUT THE INVENTION

Hereinafter, the present invention will be described in more detail by way of the following examples. However, the following examples are only for illustrating the present invention, and the scope of the present invention is not limited only to these examples.

### I. Preparation of Acellular Dermal Matrix Using Supercritical Fluid Extraction Process

### Example 1. Preparation of Acellular Dermal Matrix Using Supercritical Fluid Extraction Process

For decellularization of skin tissue, a supercritical fluid extraction process was performed.

First, the fat layer was removed from the donated patient's human skin tissue (TRB No. 20201305, Seoul Asan Medical Center). After the fat layer was removed, the epidermis was removed by treating it with 1 M NaCl (Sigma Aldrich, Cat No. S9888) under a temperature condition of 37° C. for 24 hours. After the epidermis was removed, the separated dermal layer was washed with sterilized PBS (biowest, Cat No. L0615-500) for 1 hour.

After washing, the obtained dermal tissue was placed in the extraction tank of a supercritical extraction system (SES), and supercritical fluid carbon dioxide and co-solvent ethanol were injected together into the extraction tank. Thereafter, the dermal tissue was decellularized by supercritical treatment for 1 to 3 hours under a pressure condition of 72.8 bar and a temperature condition of 31° C.

Thereafter, the decellularized dermal tissue was washed with sterilized PBS at room temperature for 24 hours. For an RTU (Ready-To-Use) type, the washed acellular dermal tissue was sealed and packaged with sterile physiological saline. In addition, for an FD (Freeze-Drying) type, the acellular dermal tissue was treated and penetrated with maltitol and frozen at −80° C. for 4 hours or more. Thereafter, it was freeze-dried for 24 hours using a freeze-dryer and then sealed and packaged. The packaged acellular dermal tissue was sterilized by performing gamma sterilization (Greenpia, 15 kGy) and then stored at room temperature until use.

### Preparation Example 1. Preparation of Acellular Dermal Matrix Sample and Observation of Appearance

As a comparison group for the acellular dermal matrix (ADM) sample prepared in Example 1 above, a human dermal ADM sample prepared according to a conventionally known method according to the detailed specifications in Table 1 below was prepared.

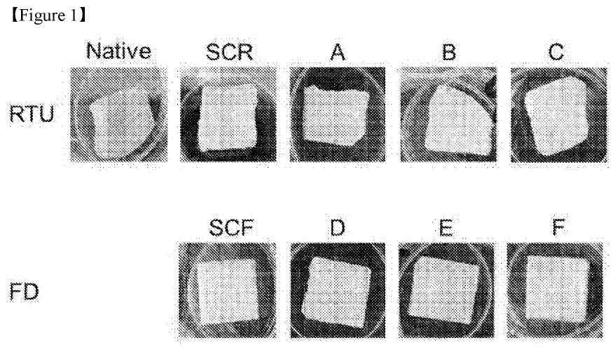

The thickness of each prepared sample was measured (Table 2), and the appearance was observed (FIG. 1). At this time, the thickness was measured using a vernier caliper to measure the thickness of three different parts of a commercially available product.

TABLE 1

| | Sample | | |
|---|---|---|---|
| No. | name | Standards and detailed specifications | Note |
| 1 | Native | RTU, g-15 kGy | Native |
| 2 | SCR | RTU, g-15 kGy (see Example 1) | Applying supercritical fluid |
| 3 | SCF | Freeze-drying, γ-15 kGy (see Example 1) | treatment process |
| 4 | A | 4 × 12 cm², RTU, E-Beam | Applying chemical treatment |
| 5 | B | 4 × 10 cm², RTU, γ-15 kGy | (surfactant treatment) or |
| 6 | C | 4 × 5 cm², RTU, E-Beam | enzymatic treatment process |
| 7 | D | 4 × 7 cm², Freeze-drying, Aceptic | |
| 8 | E | 1 × 5 cm², Freeze-drying, E-Beam | |
| 9 | F | 4 × 5 cm², Freeze-drying, EO Gas | |

TABLE 2

| | Sample | Thickness |
|---|---|---|
| No. | name | (mm) |
| 1 | Native | 3.0 |
| 2 | SCR | 3.0 |
| 3 | SCF | 2.8 |
| 4 | A | 3.0 |
| 5 | B | 2.0 |
| 6 | C | 3.0 |
| 7 | D | 3.0 |
| 8 | E | 1.0 |
| 9 | F | 3.0 |

Specifically, all dermal tissues including Native tissues were treated with gamma ray sterilization (15 kGy). At this time, an FD (Freeze-Drying) type Native sample was not included, and an RTU type Native tissue was used as an untreated control sample for all analyses. In addition, the Native value included in the analysis results of FD type samples was expressed as the value of RTU (Ready-To-Use) type Native samples.

The RTU type was prepared by double packaging the dermal samples including normal saline, followed by sterilization. In addition, the FD type was prepared by freeze-drying and double packaging the dermal sample, followed by sterilization. The FD type acellular dermal matrix samples were immersed in normal saline for about 100 minutes to hydrate them, and their appearance was observed (FIG. 1).

As shown in FIG. 1, it was confirmed that in terms of appearance, the color of the acellular dermal matrix samples (SCR and SCF) prepared in Example 1 above were similar to that of a human dermal ADM product prepared according to a conventionally known method.

### H. Evaluation of Biochemical Properties of Acellular Dermal Matrix According to Supercritical Fluid Extraction Process

### Experimental Example 1. Histological Analysis of Decellularized Dermal Tissue

For histological analysis of the decellularized dermal tissue prepared in Example 1 above, H&E (Hematoxylin & Eosin) staining was performed to analyze the histological morphology and decellularization level of the tissue. That is, the presence or absence of cell nuclei was confirmed through Hematoxylin (blue) & Eosin (red) staining, an indicator of decellularization performance. As hematoxylin, which binds to the DNA of cells, is related to the DNA quantification results, the decellularization level can be analyzed.

For H&E staining, the original tissue sample (Native) that was not subjected to the supercritical fluid treatment without removing the epidermis was used as a negative control group. The acellular dermal matrix samples (SCR and SCF) that were subjected to the supercritical fluid treatment with removing the epidermis were used as a test group. In addition, the human dermal ADM samples (A, B, C, D, E, and F) according to the detailed specifications of Table 1 were used as a comparison group.

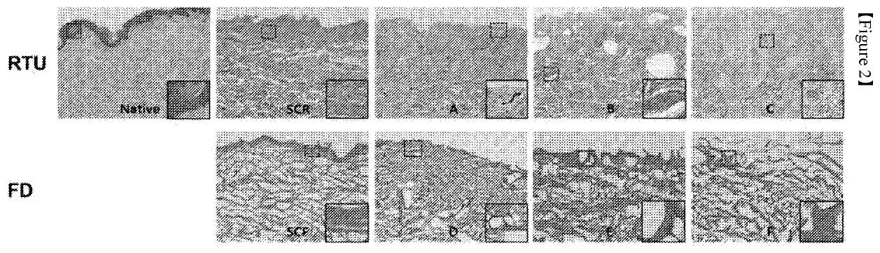

As a result of staining, it was confirmed that the nuclei were completely removed in the decellularized dermal samples SCR and SCF according to the supercritical fluid process (FIG. 2). In addition, no nuclei were observed in the C and F samples among the human dermal ADM samples. In contrast, in the A, B, D, and E samples, the Hematoxylin staining part was confirmed, indicating that decellularization was not completely achieved.

Meanwhile, it was confirmed that the preservation level of the internal structure of the tissue was the most excellent in the A and D samples. Specifically, in the case of the RTU type, the preservation level of the internal structure of the tissue was excellent in the order of A>SCR (supercritical fluid process treated sample)>C>B. In addition, in the case of the FD type, the preservation level of the internal structure of the tissue was excellent in the order of D>SCF (supercritical fluid process treated sample)>E=F.

### Experimental Example 2. Confirmation of Content of Residual DNA in Decellularized Dermal Tissue

In order to confirm the decellularization level of the decellularized dermal tissue (SCR, SCF) prepared in Example 1 above, the DNA content in the decellularized dermal sample was measured. At this time, the original tissue (Native) that was not subjected to the supercritical fluid treatment was used as a negative control group, and the acellular dermal tissue sample of Table 1 prepared according to the conventional method was used as a positive control group. Measurement of DNA content was performed on RTU (Ready-To-Use) type and FD (Feeze-Drying) type acellular dermal matrix samples.

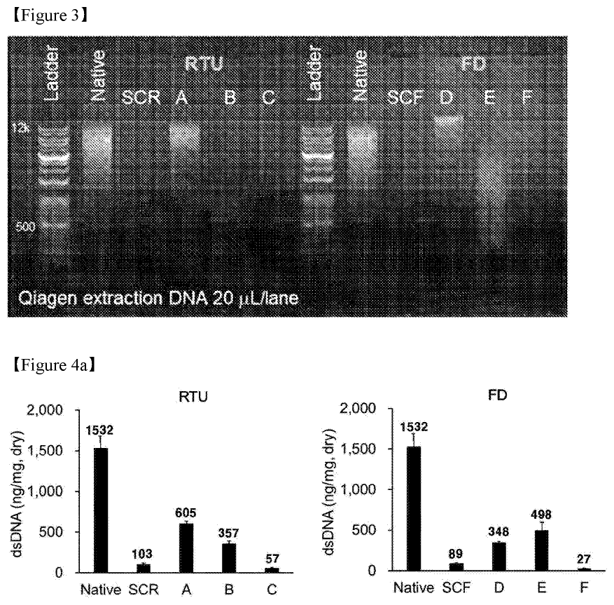

Specifically, for each sample, gDNA of each dermal tissue was extracted using the Dneasy Blood & tissue kit (QIAGEN, Cat #69506). Thereafter, electrophoresis was performed using 1% agarose gel (FIG. 3). During electrophoresis, ExcelBand™ 1 KB Plus (0.1-10 kb) DNA Ladder (SMOBIO, Cat #DM3200) was used as a size marker.

As a result of performing electrophoresis, it was confirmed that the amount of residual dsDNA in the decellularized dermal samples (SCR and SCF) according to the supercritical fluid treatment process was low compared to that of the commercially available acellular dermal matrix samples (A, B, C, D, E, and F) for both RTU and FD types. Among the acellular dermal matrix samples prepared by chemical or enzymatic treatment according to the conventional method, a DNA band was weakly observed in B, but a clear DNA band was confirmed in the remaining acellular dermal matrix samples A, D, and E. In contrast, it was confirmed that no DNA band was observed at all in SCR and SCF (FIG. 3).

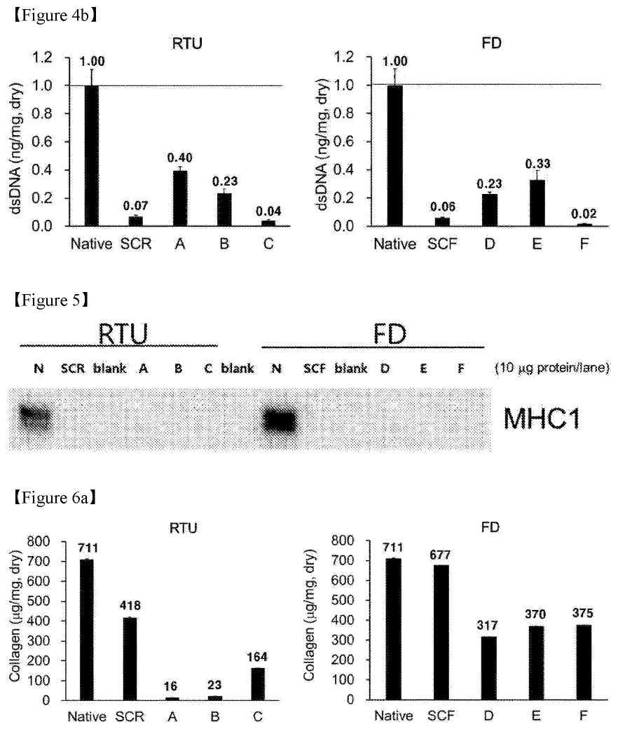

In addition, the content of residual dsDNA in the acellular dermal matrix samples was quantitatively analyzed using the Qubit dsDNA BR assay kit (Thermo, Cat #Q32853), which employs a method of measuring fluorescence intensity by adding a fluorescent dye that directly binds to a specific region of dsDNA to the extracted DNA (FIGS. 4a and 4b). At this time, all samples were freeze-dried and analyzed based on the solid mass.

As a result of the analysis, the DNA quantitative value was measured as a result similar to the sum of fragmented DNA from electrophoresis. That is, it was confirmed that similar to the electrophoresis results, the amount of residual dsDNA in the decellularized dermal samples (SCR and SCF) according to the supercritical fluid treatment process was low compared to the acellular dermal matrix samples (A, B, C, D, E, and F) prepared according to the conventional method for both RTU and FD types.

Through this, it was found that the DNA removal effect was excellent in the supercritical fluid treatment compared to the chemical treatment or enzymatic treatment process using surfactants applied in the conventional acellular dermal matrix production. In addition, the decellularization of 90% or more of the original tissue was confirmed, indicating that it has an excellent effect that can be used as a graft material without transplant rejection.

### Experimental Example 3. Confirmation of Expression Level of Immunogenic Proteins in Decellularized Dermal Tissue

In order to confirm the level of transplant rejection in the decellularized dermal tissue (SCR and SCF) prepared in Example 1 above, the expression level of immunogenic protein MHC1 in the acellular dermal matrix sample was confirmed. At this time, the acellular dermal tissue samples (A, B, C, D, E, and F) prepared by the conventional chemical treatment or enzymatic treatment process were as a positive control group, and the test was performed on acellular dermal matrix samples of RTU type and FD type.

Specifically, for each sample, proteins of each dermal tissue were extracted using RIPA lysis buffer. Thereafter, protein quantification was performed using Pierce BCA Protein Assay Kit (Thermo, Cat #23225) that employs the BCA (bicinchoninic acid) method. After protein quantification, Western blot was performed using the MHC1 (SANTA CRUZ Biotechnology, Inc., Cat #sc-55582) antibody (FIG. 5).

As a result of the experiment, it was confirmed that the immunogenic protein MHC1, a cell membrane protein, was negative in all acellular dermal matrix samples.

Through this, it was confirmed that the expression of the immunogenic protein MHC1 was negative when treated with supercritical fluid, similar to the acellular dermal tissue sample prepared according to the conventional method, indicating that it has an excellent effect that can be used as a graft material without transplant rejection.

### Experimental Example 4. Confirmation of Preservation Level of Extracellular Matrix (ECM) of Decellularized Dermal Tissue

The preservation level of the extracellular matrix components, i.e., collagen, elastin, and sGAG, etc. in the decellularized dermal tissue (SCR and SCF) prepared in Example 1 above was confirmed.

### Experimental Example 4.1. Confirmation of Collagen Content in Decellularized Dermal Tissue

In order to confirm that there was no loss of protein in the decellularized dermal tissue (SCR and SCF) prepared in Example 1 above, the collagen content was measured using the Sircol insoluble Collagen assay kit (Biocolor, Cat #S2000). At this time, the original tissue (Native) that was not subjected to the supercritical fluid treatment was used as a negative control group. The dermal tissue samples (A, B, C, D, E, and F) that were subjected to enzymatic treatment or chemical treatment using a surfactant were used as a positive control group. Each sample was freeze-dried and about 5 mg was used to measure the collagen content.

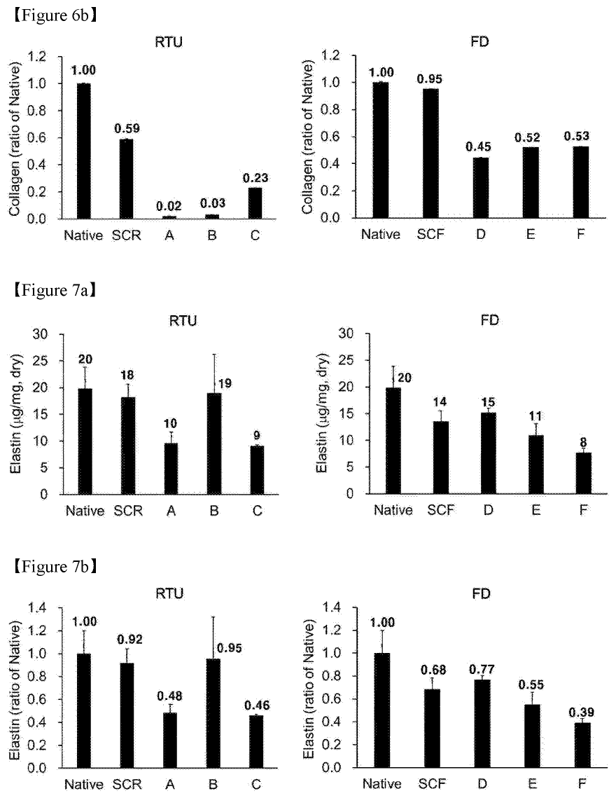

As a result of the measurement, it was confirmed that the collagen preservation rate in the decellularized dermal tissue according to the supercritical fluid process was the most excellent compared to the acellular dermal tissue sample prepared according to the conventional method based on the original tissue in both RTU and FD type samples. Specifically, it was confirmed that the collagen content was 418 μg/mg for the RTU type sample and 677 μg/mg for the FD type sample based on the dry weight of the sample. This was 58.8% and 95.2%, respectively, compared to the original tissue, indicating that the protein loss in the decellularized dermal tissue according to the supercritical fluid process was minimized (FIGS. 6a and 6b).

In contrast, it was confirmed that the collagen content in the dermal tissue decellularized by the conventional chemical treatment or enzymatic treatment process was 16 g/mg, 23 μg/mg, and 164 μg/mg for RTU type A, B, and C samples, respectively, and 317 g/mg, 370 μg/mg, and 375 μg/mg for FD type D, E, and F samples, respectively, based on the dry weight of the sample. This was only 2.25%, 3.23%, and 23.1% for RTU type A, B, and C samples, respectively, and 44.6%, 52.0%, and 52.7% for FD type D, E, and F samples, respectively, compared to the original tissue. In other words, it was found that when decellularized by the conventional chemical treatment or enzymatic treatment process, although the decellularization effect was excellent, significant protein loss occurred in the dermal tissue.

Through the above results, it was found that the decellularized dermal tissue by the supercritical fluid process treatment maintained its histological morphology and was effectively decellularized while minimizing protein loss. Accordingly, it was found that it has an excellent effect that can be utilized as a graft material.

### Experimental Example 4.2. Confirmation of Elastin Content in Decellularized Dermal Tissue

In order to confirm that there was no loss of elastin in the decellularized dermal tissue (SCR, SCF) prepared in Example 1 above, the elastin analysis was performed using the Fastin Elastin assay kit (Biocolor, Cat #F2000). At this time, the original tissue (Native) that was not subjected to the supercritical fluid treatment was used as a negative control group. The dermal tissue samples (A, B, C, D, E, and F) that were subjected to enzymatic treatment or chemical treatment using a surfactant were used as a positive control group. Each sample was freeze-dried and about 5 mg was used to measure the elastin content.

As a result of the analysis, it was confirmed that the elastin preservation rate in the decellularized dermal tissue according to the supercritical fluid process was maintained at a level equal to or higher than that of the acellular dermal tissue sample prepared according to the conventional method based on the original tissue in both RTU and FD type products. Specifically, it was confirmed that the elastin content was 18 μg/mg for the RTU type sample and 14 μg/mg for the FD type sample based on the dry weight of the sample. This was 90% and 70%, respectively, compared to the original tissue, and it was found that the elastin loss in the decellularized dermal tissue according to the supercritical fluid process was not occurred (FIGS. 7a and 7b).

In contrast, it was confirmed that the elastin content in the dermal tissue decellularized by the conventional chemical treatment or enzymatic treatment process was 10 μg/mg, 19 g/mg, and 9 μg/mg for RTU type A, B, and C samples, respectively, and 15 μg/mg, 11 μg/mg, and 8 μg/mg for FD type D, E, and F samples, respectively, based on the dry weight of the sample. This was 50%, 95%, and 45% for RTU type A, B, and C samples, respectively, and 75%, 55%, and 40% for FD type D, E, and F samples, respectively, compared to the original tissue. In other words, it was found that significant elastin loss occurred except for B and D samples.

Through the above results, it was found that the decellularized dermal tissue by the supercritical fluid process treatment maintained its histological morphology while minimizing elastin loss, and thus it has an excellent effect that can be utilized as a graft material.

### Experimental Example 5. Confirmation of Level of Growth Factor Preservation in Decellularized Dermal Tissue

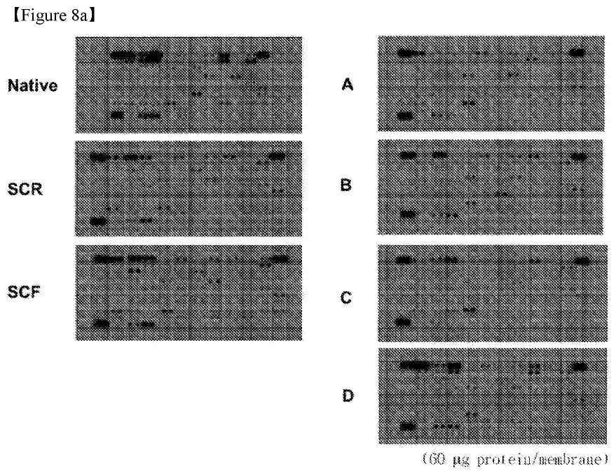

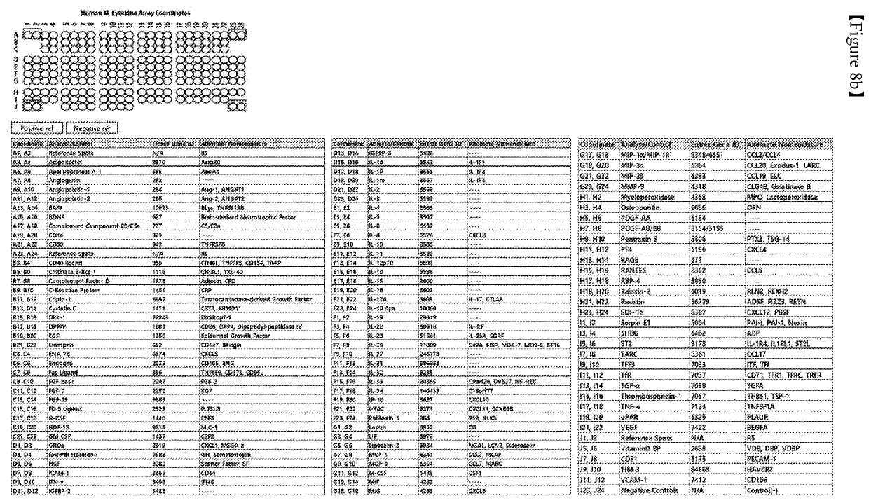

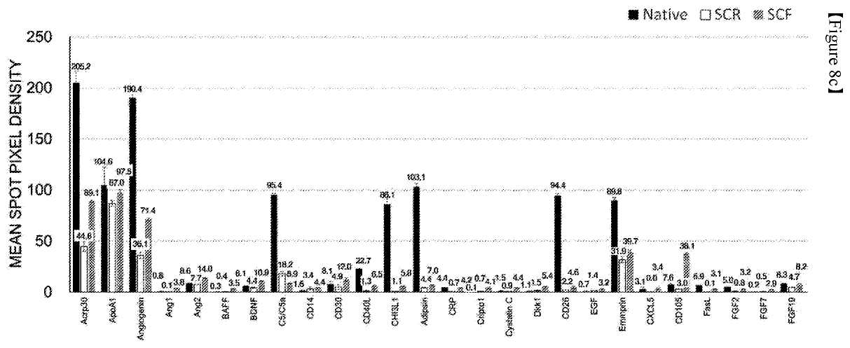

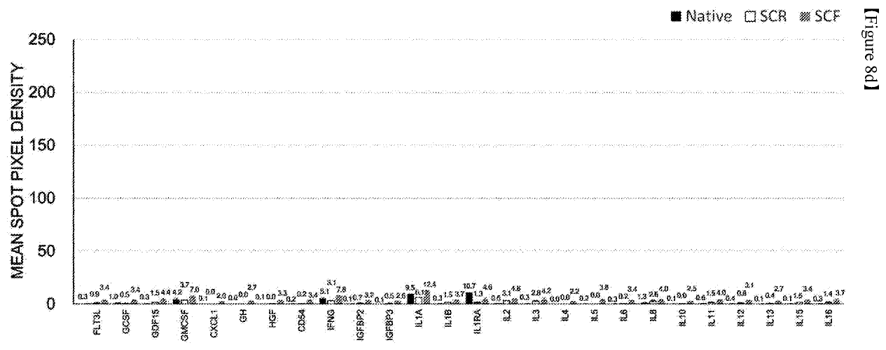

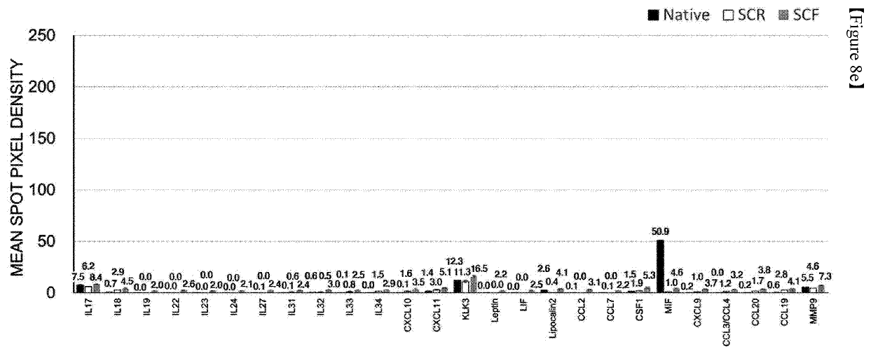

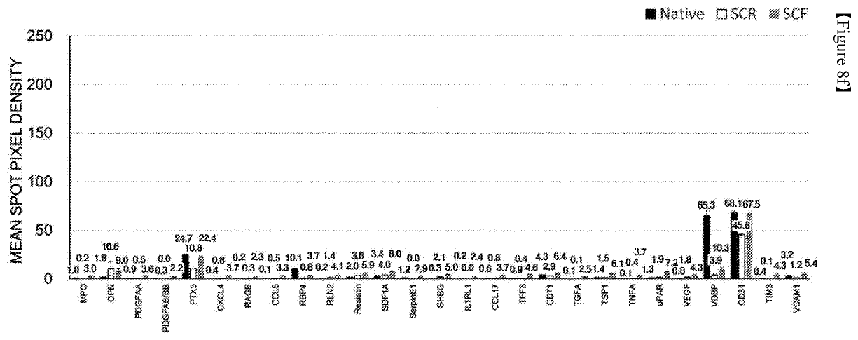

In order to confirm the level of growth factor preservation in the decellularized dermal tissue (SCR and SCF) prepared in Example 1 above, the number of cytokines expressed prominently and the expression level were confirmed using the Proteome Profiler Array, Human XL Cytokine Array Kit, and 105 Human Cytokine Array (ARY022B; R&D Systems USA) (FIGS. 8a to 8f). At this time, the original tissue (Native) that was not subjected to the supercritical fluid treatment was used as a negative control group. The dermal tissue samples (A, B, C, and D) that were subjected to enzymatic treatment or chemical treatment using a surfactant were used as a positive control group.

Specifically, the protein was extracted, separated, and purified for each sample using RIPA lysis buffer from the acellular dermal tissue samples. Thereafter, protein quantification was performed using the Pierce BCA Protein Assay Kit (Thermo, Cat #23225) that employs the BCA method. 100 mg of protein per sample was loaded equally onto each array membrane [Proteome Profiler Array, Human XL Cytokine Array Kit (ARY022B), R&D Systems USA]and analyzed.

The dot intensity values were measured using ImageJ software, and then the number of cytokines with an arbitrary intensity of 20 or higher was counted. The analysis results are summarized in Tables 3 and 4 below.

TABLE 3

| Sample | Cytokine # |
|---|---|
| name | (intensity > 20*) |
| Native | 13 |
| SCR | 5 |
| SCF | 7 |
| A | 2 |
| B | 4 |
| C | 3 |
| D | 6 |

*Mean spot pixel intensity

TABLE 4

| | Mean spot pixel intensity | | |
|---|---|---|---|
| | value for each sample | | |
| Cytokine | Native | SCR | SCF |
| Acrp30 | 205.2 | 44.8 | 89.1 |
| ApoA1 | 104.6 | 87.0 | 97.5 |
| Angiogenin | 190.4 | 36.1 | 71.4 |
| Ang1 | 0.8 | 0.1 | 3.8 |
| Ang2 | 8.6 | 7.7 | 14.0 |
| BAFF | 0.3 | 0.4 | 3.5 |
| BDNF | 6.1 | 4.4 | 10.9 |
| C5/C5a | 95.4 | 18.2 | 8.9 |
| CD14 | 1.6 | 3.4 | 4.4 |
| CD30 | 8.1 | 4.9 | 12.0 |
| CD40L | 22.7 | 1.3 | 6.5 |
| CHI3L1 | 86.1 | 1.1 | 5.8 |
| Adipsin | 103.1 | 4.4 | 7.0 |
| CRP | 4.4 | 0.7 | 4.2 |
| Cripto1 | 0.1 | 0.7 | 4.1 |
| Cystatin C | 1.5 | 0.9 | 4.4 |
| Dkk1 | 1.1 | 1.5 | 5.4 |
| CD26 | 94.4 | 2.2 | 4.6 |
| EGF | 0.7 | 1.4 | 3.2 |
| Emmprin | 89.8 | 31.9 | 39.7 |
| CXCL5 | 3.1 | 0.0 | 3.4 |
| CD105 | 7.6 | 3.0 | 38.1 |
| FasL | 6.9 | 0.1 | 3.1 |
| FGF2 | 5.0 | 0.8 | 3.2 |
| FGF7 | 0.2 | 0.5 | 2.9 |
| FGF19 | 8.3 | 4.7 | 8.2 |
| FLT3L | 0.3 | 0.9 | 3.4 |
| GCSF | 1.0 | 0.5 | 3.4 |
| GDF15 | 0.3 | 1.5 | 4.4 |
| GMCSF | 4.2 | 3.7 | 7.0 |
| CXCL1 | 0.1 | 0.0 | 2.0 |
| GH | 0.0 | 0.0 | 2.7 |
| HGF | 0.1 | 0.0 | 3.3 |
| CD54 | 0.2 | 0.2 | 3.4 |
| IFNG | 5.1 | 3.1 | 7.8 |
| IGFBP2 | 0.1 | 0.7 | 3.2 |
| IGFBP3 | 0.1 | 0.5 | 2.6 |
| IL1A | 9.5 | 6.1 | 12.4 |
| IL1B | 0.3 | 1.5 | 3.7 |
| IL1RA | 10.7 | 1.3 | 4.6 |
| IL2 | 0.6 | 3.1 | 4.8 |
| IL3 | 0.3 | 2.8 | 4.2 |
| IL4 | 0.0 | 0.0 | 2.2 |
| IL5 | 0.2 | 0.0 | 3.8 |
| IL6 | 0.3 | 0.2 | 3.4 |
| IL8 | 1.3 | 2.8 | 4.0 |
| IL10 | 0.1 | 0.0 | 2.5 |
| IL11 | 0.6 | 1.5 | 4.0 |
| IL12 | 0.4 | 0.8 | 3.1 |
| IL13 | 0.1 | 0.4 | 2.7 |
| IL15 | 0.1 | 1.6 | 3.4 |
| IL16 | 0.3 | 1.4 | 3.7 |
| IL17 | 7.5 | 6.2 | 8.4 |
| IL18 | 0.7 | 2.9 | 4.5 |
| IL19 | 0.0 | 0.0 | 2.0 |
| IL22 | 0.0 | 0.0 | 2.6 |
| IL23 | 0.0 | 0.0 | 2.0 |
| IL24 | 0.0 | 0.0 | 2.1 |
| IL27 | 0.1 | 0.0 | 2.4 |
| IL31 | 0.1 | 0.6 | 2.4 |
| IL32 | 0.6 | 0.5 | 3.0 |
| IL33 | 0.1 | 0.8 | 2.5 |
| IL34 | 0.0 | 1.5 | 2.9 |
| CXCL10 | 0.1 | 1.6 | 3.5 |
| CXCL11 | 1.4 | 3.0 | 5.1 |
| KLK3 | 12.3 | 11.3 | 16.5 |
| Leptin | 0.0 | 0.0 | 2.2 |
| LIF | 0.0 | 0.0 | 2.5 |
| Lipocalin2 | 2.6 | 0.4 | 4.1 |
| CCL2 | 0.1 | 0.0 | 3.1 |
| CCL7 | 0.1 | 0.0 | 2.2 |
| CSF1 | 1.5 | 1.9 | 5.3 |
| MIF | 50.9 | 1.0 | 4.6 |
| CXCL9 | 0.2 | 1.0 | 3.7 |
| CCL3/CCL4 | 0.0 | 1.2 | 3.2 |
| CCL20 | 0.2 | 1.7 | 3.8 |
| CCL19 | 0.6 | 2.8 | 4.1 |
| MMP9 | 5.5 | 4.6 | 7.3 |
| MPO | 1.0 | 0.2 | 3.0 |
| OPN | 1.8 | 10.6 | 9.0 |
| PDGFAA | 0.9 | 0.5 | 3.6 |
| PDGFAB/BB | 0.3 | 0.0 | 2.2 |
| PTX3 | 24.7 | 10.8 | 22.4 |
| CXCL4 | 0.4 | 0.8 | 3.7 |
| RAGE | 0.2 | 0.3 | 2.3 |
| CCL5 | 0.1 | 0.5 | 3.3 |
| RBP4 | 10.1 | 0.8 | 3.7 |
| RLN2 | 0.2 | 1.4 | 4.1 |
| Resistin | 2.0 | 3.6 | 5.9 |
| SDF1A | 3.4 | 4.0 | 8.0 |
| SerpinE1 | 1.2 | 0.0 | 2.9 |
| SHBG | 0.3 | 2.1 | 5.0 |
| IL1RL1 | 0.2 | 0.0 | 2.4 |
| CCL17 | 0.6 | 0.8 | 3.7 |
| TFF3 | 0.9 | 0.4 | 4.6 |
| CD71 | 4.3 | 2.9 | 6.4 |
| TGFA | 0.1 | 0.1 | 2.5 |
| TSP1 | 1.4 | 1.5 | 6.1 |
| TNFA | 0.1 | 0.4 | 3.7 |
| uPAR | 1.3 | 1.9 | 7.2 |
| VEGF | 0.8 | 1.8 | 4.3 |
| VDBP | 65.3 | 3.9 | 10.3 |
| CD31 | 68.1 | 45.6 | 67.5 |
| TIM3 | 0.4 | 0.1 | 4.3 |
| VCAM1 | 3.2 | 1.2 | 5.4 |

As a result of performing the cytokine array, it was confirmed that the growth factor preservation rate was equivalent to that of the acellular dermal tissue D sample prepared according to the conventional method. In particular, it was found that among the RTU type samples, the growth factor preservation rate of the acellular dermal matrix sample (SCR) according to the supercritical fluid process of the present invention was the most excellent.

Meanwhile, as a result of comparing the growth factor preservation rate between the D sample prepared according to the conventional freeze-drying type method and the SCF sample according to the present invention, although there was a difference in whether gamma sterilization was performed, it was found that the growth factor preservation rate of the SCF acellular dermal matrix of the present invention, which was decellularized by the supercritical fluid treatment process, was superior even in the same freeze-dried type sample.

### Experimental Example 6. Cytotoxicity Evaluation

The cytotoxicity of the decellularized dermal tissue (SCR and SCF) prepared in Example 1 above was evaluated. Specifically, the cytotoxicity evaluation was performed using the L929 mouse fibroblast cell line known in the art, and with the cell number of 1×10⁴ cells/well. In addition, the cytotoxicity evaluation was performed according to the tissue elution method (applying the KCL accredited testing agency protocol) in which the tissue is immersed in a cell culture solution at 37° C. for 24 hours to elute the internal material of the tissue into the culture solution. At this time, if the survival rate was reduced to less than 70% of the blank test solution according to the above-mentioned accredited testing agency standards, it was evaluated as potentially cytotoxic.

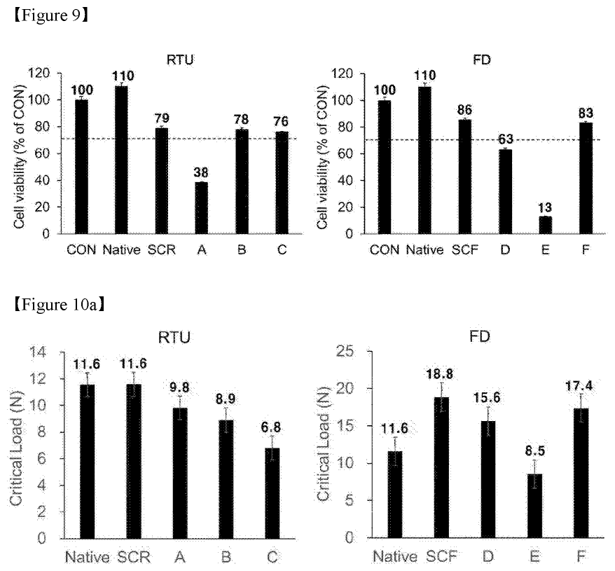

As a result of the cytotoxicity evaluation, it was found that the cytotoxicity of the acellular dermal matrix samples (SCR and SCF) according to the supercritical fluid process of the present invention was significantly lower than that of the acellular dermal tissue samples (A, B, C, D, E, and F) prepared by the conventional chemical treatment or enzymatic treatment process (FIG. 9).

Through the above results, it was found that the decellularized dermal tissue by the supercritical fluid process treatment maintained its histological morphology without cytotoxicity, and thus it has an excellent effect that can be utilized as a graft material.

### III. Evaluation of Physicochemical Properties of Acellular Dermal Matrix According to Supercritical Fluid Extraction Process

### Experimental Example 7. Measurement of Critical Load and Elasticity of Decellularized Dermal Tissue Through Supercritical Fluid Extraction Process

The critical load and elasticity were measured for the acellular dermal tissue (SCR and SCF) prepared in Example 1 above. The critical load and elasticity of the decellularized dermal tissue through the supercritical fluid extraction process were measured using a universal tensile machine (EZ-x model (Shimadzu co.)). At this time, the samples with the size of 1×1 cm² were tested. For the test sample, the original tissue (Native) that was not subjected to the supercritical fluid treatment was used as a negative control group. The dermal tissue samples (A, B, C, D, E, and F) that were subjected to enzymatic treatment or chemical treatment using a surfactant were used as a positive control group. Meanwhile, prior to the measurement of critical load and elasticity, each sample was rehydrated with PBS for 100 minutes, and the moisture on the tissue surface was removed with gauze just before the measurement.

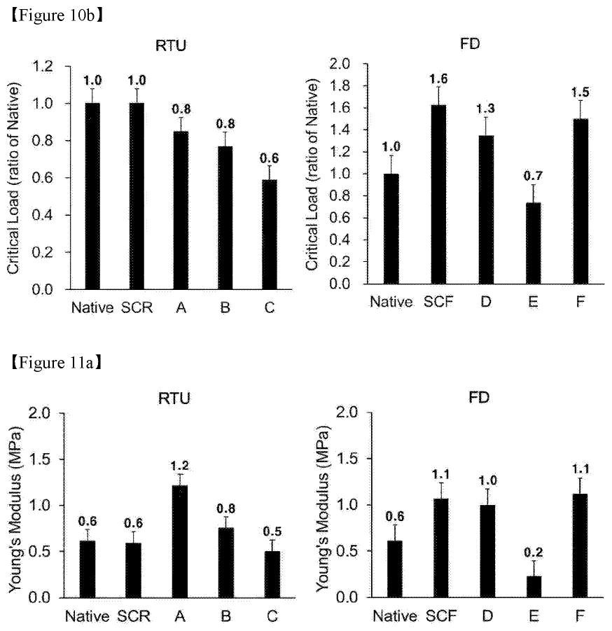

As a result, it was confirmed that the critical load for the decellularized dermal tissue (SCR) according to the RTU type supercritical fluid process was 11.6 N, which was almost completely maintained compared to the original tissue, unlike the acellular dermal tissue sample A (9.8 N), sample B (8.9 N), and sample C (6.8 N) prepared according to the conventional method. In particular, it was confirmed that the critical load for the decellularized dermal tissue (SCF) according to the FD type supercritical fluid process was 18.8 N, which was about 1.6 times higher than the original tissue (11.6 N), and it showed a critical load level equivalent to or higher than the acellular dermal tissue sample D (15.6 N), sample E (8.5 N), and sample F (17.4 N) prepared according to the conventional method (FIGS. 10a and 10b).

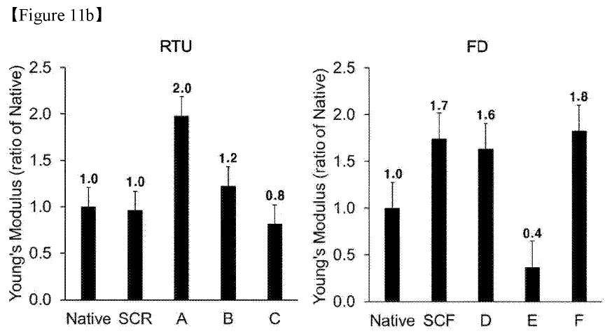

The Young's modulus of the RTU type SCR sample according to the present invention was also measured to be 0.6 MPa, which was equal to the Young's modulus of the original tissue sample, confirming that the elasticity was almost completely maintained compared to the original tissue. In addition, it was confirmed that the Young's modulus for the FD type SCF sample was 1.1 MPa, which was about 1.8 times higher than the Young's modulus of the original tissue sample (0.6 MPa), and it showed a Young's modulus level equivalent to or higher than that of the acellular dermal tissue sample D (1.0 MPa), sample E (0.2 MPa), and sample F (1.1 MPa) prepared according to the conventional method (FIGS. 11a and 11b).

1. An acellular dermal tissue graft, wherein a critical load of the acellular dermal tissue is 11 to 19 N, and a Young's modulus of the acellular dermal tissue is 0.5 to 1.2 MPa.

2. The acellular dermal tissue graft according to claim 1, wherein the acellular dermal tissue has any one characteristic selected from the following characteristics based on dry weight:
an amount of residual DNA of 80 to 110 ng/mg;
a collagen content of 400 to 700 μg/mg; and
an elastin content of 13 to 19 μg/mg.

3. The acellular dermal tissue graft according to claim 1, wherein the acellular dermal tissue has any one characteristic selected from the following characteristics compared to the original tissue:
an amount of residual DNA of 5 to 8%;
a collagen content of 55 to 99%;
an elastin content of 65 to 95%;
a critical load of 90 to 165%; and
a Young's modulus of 80 to 200%.

4. The acellular dermal tissue graft according to claim 1, wherein the acellular dermal tissue has any one characteristic selected from the following characteristics compared to the original tissue:
an adiponectin content of 22 to 44%;
an apolipoprotein A1 content of 84 to 94%;
an angiogenin content of 19 to 38%;
an angiopoietin-2 content of 90 to 99%;
a brain-derived neurotrophic factor (BDNF) content of 73 to 99%;
a complement component C5/C5a content of 9 to 19%;
a CD30 content of 59 to 99%;
a CD40 ligand content of 6 to 28%;
a CD26 content of 2 to 5%;
an Emmprin content of 36 to 44%;
a CD105 content of 41 to 99%;
a Fas ligand content of 2 to 45%;
a FGF-2 content of 15 to 64%;
a FGF19 content of 56 to 98%;
an IFN-γ content of 63 to 99%;
an IL-1A (alpha) content of 65 to 99%;
an IL-IRA (alpha) content of 12 to 43%;
an IL-17 content of 82 to 99%;
a kallikrein (KLK) 3 content of 92 to 99%;
a MIF (Macrophage Migration Inhibitory Factor) content of 2 to 9%;
a MMP (Matrix Metalloproteinase Protein) 9 content of 83 to 99%;
a pentraxin (PTX) 3 content of 44 to 91%;
a retinol binding protein (RBP) 4 content of 8 to 36%;
a vitamin D binding protein (VDBP) content of 6 to 16%; and
a CD31 content of 67 to 99%.

5. The acellular dermal tissue graft according to claim 1, wherein the acellular dermal tissue is prepared by a preparation method comprising the step of extracting with a supercritical fluid.

6. The acellular dermal tissue graft according to claim 5, wherein a solvent used as the supercritical fluid is carbon dioxide.

7. The acellular dermal tissue graft according to claim 5, wherein the supercritical fluid further comprises ethanol as a co-solvent.

8. The acellular dermal tissue graft according to claim 5, wherein the extraction is performed under a temperature condition of 30° C. to 40° C.

9. The acellular dermal tissue graft according to claim 5, wherein the extraction is performed under a pressure condition of 50 bar to 400 bar.

10. The acellular dermal tissue graft according to claim 5, wherein the extraction is performed for 1 to 3 hours.

11. The acellular dermal tissue graft according to claim 5, wherein the step of separating an epidermal layer and a dermal layer is included before the step of extracting with a supercritical fluid.

12. The acellular dermal tissue graft according to claim 5, wherein the preparation method comprises any one of the following steps after the step of extracting with a supercritical fluid:
washing a dermal tissue with a phosphate buffer;
freeze-drying the dermal tissue; and
sterilizing the dermal tissue.

13. A method for preparing an acellular dermal tissue graft, comprising the steps of:
a) separating skin tissue separated from a subject into an epidermal layer and a dermal layer;
b) extracting the separated dermal layer with a supercritical fluid; and
c) washing the dermal layer extracted with a supercritical fluid with a phosphate buffer.

14. The preparation method according to claim 13, wherein the supercritical fluid in step b) further comprises a co-solvent.
